(19) United States
(12) Patent Application Publication
(10) Pub. No.: US 2021/0030917 A1
(43) Pub. Date: Feb. 4, 2021

(54) NANOFIBROUS ADHESION BARRIER

(71) Applicants: Esra KARACA, Bursa (TR); Rabia Gözde ÖZALP, Bursa (TR); Serife SAFAK, Bursa (TR); INOVENSO TEKNOLOJI LIMITED SIRKETI, Istanbul (TR)

(72) Inventors: Esra KARACA, Bursa (TR); Rabia Gözde ÖZALP, Bursa (TR); Serife SAFAK, Bursa (TR)

(21) Appl. No.: 16/968,216

(22) PCT Filed: Nov. 1, 2018
(86) PCT No.: PCT/TR2018/050649

§ 371 Date: Aug. 7, 2020

(30) Foreign Application Priority Data
Feb. 12, 2018 (TR) 2018/01865

(51) Int. Cl. A61L 15/28; D01D 5/00; D01D 5/38; D01D 5/28

(52) U.S. Cl. CPC A61L 15/28; D01D 5/003; A61L 2400/12; D01D 5/28; D01D 5/38

(57) ABSTRACT

The present disclosure relates to adhesion barriers used in the biomedical field. Disclosed in particular is a nanofibrous mat suitable for use as an adhesion barrier in the biomedical field and obtained by the electrospinning method from a mixture of hyaluronic acid (HA) and sodium alginate (NaAlg) polymer solutions.

### TECHNICAL FIELD

The invention relates to adhesion barriers used in the biomedical field. The invention relates in particular to a nanofibrous mat suitable for use as an adhesion barrier in the biomedical field obtained by electrospinning method from a mixture of hyaluronic acid (HA) and sodium alginate (NaAlg) polymer solutions.

### THE PRIOR ART

Adhesions are described as abnormal adhesions that are not normally associated with each other in the intra-abdominal region, and that the organs surrounded by the serous membrane are involved with each other's and/or adjacent organs following injury or surgical operations. The main causes of adhesions are surgical procedures. Adhesions are common after thoracal, heart and abdominal operations. Postoperative intraabdominal adhesion formation rates are between 64% and 97%. Abdominal adhesions, which are one of the most important problems of both surgeons and patients, lead to chronic abdominal and pelvic pain, organ obstructions (bowel, ovarian tubule, kidney drainage channels, etc.) and functional disorders. As a result, it causes new operations to be performed. In surgical operations, it t is seen that adhesions are responsible for one-third of all intestinal obstructions and two-thirds of small intestinal obstructions [1-5]. For complications due to adhesions, only 400,000 adhesion operations are performed annually in the USA. Adhesion opening operations are long-running operations. During these operations, anesthesia and the length of stay at the hospital are prolonged. Therefore, prevention of intra-abdominal adhesion is a very important issue during surgical interventions [6, 7].

The main approaches proposed in the literature to prevent or reduce adhesion are divided into three categories: the development of surgical techniques, the use of anti-adherence drugs, and the separation of tissues in the healing process. The basic surgical principles that all surgeons must apply in order to prevent adhesion are to reduce surgical trauma as much as possible, to avoid unnecessary and excessive manipulations, to remove foreign bodies and dead tissues, to prevent dryness due to inadequate blood supply and loss of water in the tissues and to keep bacterial invasion to a minimum level. However, considering the adhesion-forming nature of the intra-abdominal region to protect the organism during the healing process, it is suggested that the therapies and technological developments to be made with the surgical technique may not prevent adhesion formation but only reduce it [3, 4]. Drugs used to prevent adhesion are either directed to inflammatory processes or to agents that cause adhesion (infection, endotoxin, exudate, etc.). The drug should be specific to adhesions and not affect normal wound healing. However, the clinical and experimental efficacy of these drugs is questionable and has side effects such as adverse effects on the immune system and delayed wound healing [7]. Another method of separating tissues from each other during the healing process is the use of adhesion barriers. Adhesion barriers allow the surfaces in the injured intra-abdominal region to be separated from each other and freely heal and thus prevent the formation of adhesion. Today, physical barriers used as adhesion inhibitors are divided into two main groups as liquid barriers and membrane barriers. These barriers are often used with a mesh material. Composite mesh structures consisting of a combination of mesh and adhesion barriers are also available. However, these materials are very expensive and cannot be used in any part of the body [4, 5].

An ideal adhesion barrier should not affect wound healing, be non-reactive, be effective in the presence of body fluids and blood, be easy to use, and be biodegradable. Also, it should not cause infection and inflammation, should be antibacterial, be stable in the initial phase of adhesion formation, then metabolize and be economical. Adhesion barriers which have recently been developed and found to be the most widely used in clinical practice are the oxide regenerated cellulose membrane (Interceed®), e-polytetrafluoroethylene membrane (Gore-tex®) and carboxymethyl cellulose/hyaluronic acid membrane (Seprafilm®). The first two are used only in gynecology, and the latter is widely used both in general surgery and in gynecology. However, the existing barriers, it requires special skills to use, despite its beneficial effects in preventing the adhesion, to give rise to complications, they cannot be used in every region and most importantly, their use is limited because they are expensive (one of them is 1.200 TL on average, and 2-3 units are used in each operation). In addition to being expensive, other disadvantages are to lead to the separation of blood vessels, the risk of abscess formation, the tendency to break when sharp edges are bent due to film structures, and the difficulty of applying them to tissue [1-8].

A lot of material was used to prevent adhesion formation until today, but it has not been precisely shown that no one blocks the intra-abdominal adhesion. Studies continue to reduce or prevent intra-abdominal adhesions with used materials and with the products put forward constitute the million dollar health market.

The natural polymers hyaluronic acid (HA) and sodium alginate (NaAlg) are used in mixture with different polymers or purely as a gel, film, membrane or fiber/nanofiber in the biomedical field. Previously, however, no nanofibrous mat was produced by electrospinning from the HA/NaAlg polymer mixture. Also, no nanofibrous mats have been produced from these polymers, either alone or in admixture, for use as an adhesion barrier.

The developments known in the art concerning the subject are given below.

The patent with the publication number EP2598180B1 relates to “Hyaluronic acid based hydrogel and its use in surgery”. The present invention relates to hydrogels based on hyaluronic acid-based derivatives which are more resistant to chemical and enzymatic degradation than hyaluronic acid alone. Hyaluronic acid-based hydrogel, in various surgeries, has an optimal use for example for injection into bone fractures or cavities and for the production of prosthetic coatings in orthopedic surgery, as fillers in cosmetic and maxillofacial surgery, and as an anti-adhesion barrier in abdominal and abdominal/pelvic surgery and in the general surgery with the prevention of postoperative adhesions.

The patent with the publication number EP1975284B1 relates to “the electrospinning apparatus for serial production of nanofibers”. The invention refers to an electro-photographic apparatus having electrical stability and an improved nozzle blocks.

The patent with the publication number TR 2013 13417 relates to “Coaxial nanofibrous mats with plant extract”. For the release of plant extracts, which are natural bioactive agents, mats are produced from coaxial biopolymer nanofibers trapped in plant extracts in their regions. Nanofibrous mats produced from natural or synthetic biopolymers with plant extracts exhibiting antimicrobial properties thanks to the phenolic components they contain, have remedial effects and can be used as tissue scaffold or drug release system. Biologically compatible and degradable nanofibrous mats capable of releasing antioxidant and antimicrobial plant extracts, are produced by coaxial electrophoresis.

As a result, due to the above-mentioned negativities and the inadequacy of the existing solutions, an improvement in the technical field has been required.

### BRIEF DESCRIPTION OF THE INVENTION

The present invention is concerned with a nanofiber adhesion barrier which meets the above-mentioned requirements, removes all disadvantages and adds some additional advantages.

The primary object of the invention is to obtain a nanofibrous mat from a mixture of hyaluronic acid (HA) and sodium alginate (NaAlg) polymer solutions suitable for use as an adhesion barrier in the biomedical field.

The invention aims to provide a nanofibrous mat by using the electrospinning method from a mixture of hyaluronic acid (HA) and sodium alginate (NaAlg) polymer solutions.

One object of the invention is to provide an alternative product that is easier and more efficient to use than commercial adhesion barriers used in the market.

Another object of the invention is to provide a nanofibrous product produced by electrospinning from sodium alginate and hyaluronic acid polymers, having the potential to inhibit/reduce adhesion by being used during intra-abdominal surgery, thus reducing post-operative complications and providing less costly alternatives to commercially available products.

The invention is a nanofibrous mat obtained from the hyaluronic acid and sodium alginate polymer mixture, suitable for use as an adhesion barrier in the biomedical field to fulfill the above-mentioned purposes.

The method of producing said nanofibrous mat to realize the objects of the invention comprises the steps of;
The preparation of the hyaluronic acid solution in the solvent,
The preparation of the aqueous sodium alginate solution,
The mixing of the two solutions prepared,
The application of the electrospinning method to the mixture solution,
The cross-linking of the obtained nanofibrous mat.

In order to realize the objects of the invention, NaOH/Dimethyl sulfoxide or NaOH/Dimethylformamide as solvent are used. In the NaOH/Dimethyl sulfoxide or NaOH/Dimethylformamide solvent, the mixing ratio between NaOH and Dimethyl sulfoxide or Dimethylformamide is 4/1 by volume

In order to realize the objects of the invention, the concentration of hyaluronic acid solution prepared in the solvent is in the range of 8-15% by weight/volume %. The concentration of sodium alginate solution prepared in pure water is in the range of 1-4% by weight/weight %.

To accomplish the objects of the invention, said hyaluronic acid solution and sodium alginate solution are mixed at ratios of 1/1-10/1 by volume.

In order to realize the objects of the invention, the cross-linking process comprises the steps that,
1-ethyl-3-(3-imethylaminopropyl) carbodiimide hydrochloride, dissolving in a solvent,
The dissolution of N-hydroxysuccinimide or divinyl sulfone in a solvent,
The mixing of the two solutions prepared by volume of 1/1-3/1 ratio,
Submerging of the nanofibrous mat into the resulting mixture solution and waiting at room temperature for 24 hours,
Agitation of the nanofibrous mats removed from the mixture solution in ethanol,
Leave to dry in an incubator at 37° C. for 12 hours.

To accomplish the objects of the invention, said 1-ethyl-3-(3-imethylaminopropyl) carbodiimide hydrochloride, N-hydroxysuccinimide or divinyl sulfone is 50-100 mM. Ethanol or methanol is used as the solvent.

In order to fulfill the above-mentioned objects, the invention is a nanofibrous mat obtained by electrospinning from a mixture of hyaluronic acid and sodium alginate polymer suitable for use as an adhesion barrier in the biomedical field.

The structural and characteristic features of the invention and all advantages thereof will be more clearly understood by means of the following figures and detailed description which are given by referring to these figures. For this reason, the evaluation should be done taking these forms and detailed explanation into consideration.

### FIGURES FOR BETTER UNDERSTANDING OF THE INVENTION

FIG. 1: Is the Scanning Electron Microscopy (SEM) views of the nanofibrous mats ((a) 2/1 mixture ratio, (b) 3/1 mixture ratio, (c) 5/1 mixture ratio) obtained from the 12% HA/2% NaAlg mixture solution.

FIG. 2: Is the EDC/NHS cross-linking process views. ((a) the samples given to the EDC/NHS solutions, (b) the samples waiting for 24 hours, (c) after the incubator)

FIG. 3: Is the view of the water resistance test of the nanofibrous mat before the cross-linking process.

FIG. 4: Is the view of the water resistance test of the nanofibrous mat after the cross-linking process.

FIG. 5: Is the view of Scanning Electron Microscopy (SEM) views of the nanofibrous mat after the cross-linking process with EDC/NHS. ((a) 50 mM/100 mM, (b) 70 mM/100 mM, (c) 80 mM/100 mM, (d) 100 mM/100 mM)

FIG. 6: It is a schematic view showing the insertion of the adhesion barrier into the tissue.

FIG. 7: It is a schematic view of the electrospinning process.

FIG. 8: Is the view of the formation of inflammation (I), fibrosis (F), and neovascularization (N) for HA/NaAlg nanofibrous mat.

FIG. 9: Is the view of the formation of inflammation (I), fibrosis (F), and neovascularization (N) for HA/CMC/NaAlg nanofibrous mat

FIG. 10: Is the view of the collagen fibril formation for HA/NaAlg nanofibrous mat.

FIG. 11: Is the view of the collagen fibril formation for HA/CMC/NaAlg nanofibrous mat

### DESCRIPTION OF PARTS REFERENCE

1. High voltage power supply
5. Feeding unit
10. Grounded collector
15. Liquid polymer
20. Cone form

### DETAILED EXPLANATION OF THE INVENTION

In this detailed explanation, the inventive nanofibrous adhesion barrier and its preferred embodiments are described only for a better understanding of the subject and without forming any restrictive effect.

The invention relates to a nanofibrous mat obtained by electrospinning a mixture of hyaluronic acid (HA) and sodium alginate (NaAg) polymers, suitable for use as an adhesion barrier in the biomedical field. In Table 1 below, the raw materials used to obtain the inventive nanofibrous mat are given.

TABLE 1
Raw materials used to obtain the nanofibrous mat
Raw material

Hyaluronic acid (HA)
Sodium alginate (NaAlg)
Sodium hydroxide (NaOH)
Dimethyl sulfoxide (DMSO)
Pure water
1-ethyl-3-(3-imethylaminopropyl) carbodiimide hydrochloride (EDC)
N-hydroxy succinimide (NHS)
Ethanol

Hyaluronic acid (HA) is high molecular weight, highly water-absorptive, antitoxic, biocompatible and biodegradable polymer. It is used in cosmetics, biomedical and food industries. Its ability to absorb water, high viscoelasticity and non-toxic properties due to high molecular weight and negatively charged nature make it possible to use HA in cosmetic, biomedical and food industries. Due to its biocompatibility and biodegradability, HA polymer has been specially found in tissue engineering applications in gel and/or film [26, 28-31].

Due to the water retention properties of the HA polymer, it is not possible to produce nanofibers in a conventional electrospinning unit. There are various studies in the literature regarding the usability of HA nanofibrous mats produced by different methods as tissue scaffolds and wound covers [32-38].

The hyaluronic acid (HA) from the glucose aminoglycan polymer is a long chain polysaccharide first isolated from the transparent fluid of the retina in the eye. It is composed of repeating disaccharide units occurred by glycosidic linkage β-1,3 and β-1,4 of the N-acetyl-D-glucosamine and D-glucuronic acid. The molecular structure of hyaluronic acid is given below.

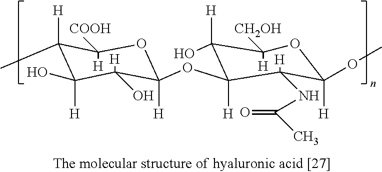

Hyaluronic acid (HA) is one of the major components of the extracellular space between cells in various living organisms such as cartilage, joint fluid, skin and umbilical cord. It can be purified from rooster swallow, baby cord and some other animal sources. In addition, it can be obtained from bacteria by fermentation and isolation methods. It has been noted that hyaluronic acid does not cause any allergic reaction in any way [25-27].

Sodium alginate (NaAlg) is a biocompatible polymer that has the ability to absorb wounds, stop the bleeding, and have high moisture absorption and gelling ability. The specific properties of sodium alginate that facilitate wound healing, high moisture absorption and ion exchange abilities, excellent biocompatibility and bleeding inhibitor properties make it a unique raw material in the production of high absorbent wound dressing [42]. Alginates have been used in food, pharmaceutical, medical, textile and paper industries for many years. Recently, its use has increased, especially in biomedical and medical fields.

Alginate is a natural polysaccharide derived from brown seaweed and on the cell walls of these mosses, it is present as calcium, magnesium and sodium salts of alginic acid. While the calcium and magnesium salts of alginate are insoluble in water, the sodium salt is soluble in water. Sodium alginate is defined as a block copolymer because its polymeric structure is formed by the incorporation of two types of monomeric acids as blocks into α-L-guluronic acid (G) and R-D-mannuronic acid (M). The molecular structure of sodium alginate is given below.

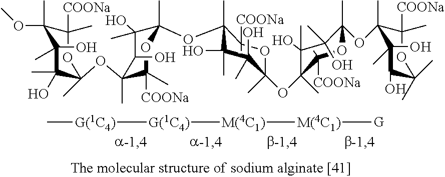

In the chemical formula of alginate, which is structurally similar to cellulose, unlike cellulose, the COOH group is replaced by the CH₂OH group [39, 40].

The most important feature of alginates is the reaction with polyvalent cations to convert them into hydrogels resulting in ion exchange. In particular, a three-dimensional networked gel structure is formed, in which the Ca+2 ions are replaced by Na+1 ions in the alginate molecule and attached to the carboxylate groups [42].

The alginate polymer has a long and rigid chain structure. Due to the rigid chain structure and high electrical conductivity, it is quite difficult to subject aqueous sodium alginate solutions to electrocution alone. In the literature, alginate nano particles produced by electrospinning in the presence of ancillary polymers are used as wound dressing [43-45], tissue scaffold [46-48] and drug release system [49], there are some studies about its use.

Nanofibrous mats are the materials proven in tissue engineering due to its flexibility, large surface area, nano-porous structure, oxygen permeability, non-bacterial permeability, similarity to natural tissue, favorable cell growth and be inexpensive of its production.

The concept of nanofiber refers to fibers with diameters less than one micron. Biomedical applications are one of the most applied areas of nanofibers. Nanofiber materials are used in many places such as medical prostheses, artificial veins and organ applications, wound covers, drug distribution systems, tissue scaffolds, skin care products. The large surface area and nano-porous structure of the nanofibrous mats provide oxygen and air permeability while exhibiting barrier properties against bacteria [9-15]. Morphologically, the mats obtained by electrospinning are very similar to the natural human extracellular matrix (ECM). Therefore, it can be used as a tissue scaffold in cell culture and tissue engineering applications. Electrospinning method is the most studied nanofiber production technique in recent years. Nanofibrous mats obtained by the electrospinning method are very convenient for cell growth and the formation of three-dimensional cellular colonies because of the attainment of very low fiber diameters. Thus, in order to accelerate or promote the formation of new cellular structures, nanofibrous mats produced by electrospinning from various polymers are used as wound covers [16, 17], drug release systems [18-20], tissue scaffolds [21-24].

The electrospinning process is based on the principle that the electrically charged liquid polymer (15) is positioned in continuous fiber form on a grounded surface [59, 60]. There are basically 4 main elements in an electrospinning mechanism.
High voltage power supply (1)
Feeding unit (jet, syringe, metal needle, etc.) (5)
Grounded collector (plates, cylinder, disc, drum, etc.) (10)
a viscous polymer in liquid form (melt or solution) (15)

In FIG. 7, a schematic representation, components, and steps of the electrospinning process are given.

The liquid polymer (15) in melt or solution is fed from a capillary tube. By means of a high voltage power supply (1), very high voltages are applied to the polymer solution. Thus, the surface of the solution droplet suspended at the tip of the needle is electrically charged. As the applied voltage increases, the polymer droplet receives the cone form (Taylor cone) (20). When the voltage reaches a critical value and the push forces of the charges in the droplet absorb the surface tension forces a thin jet is launched from the tail of the Taylor cone (20) and the jet travels from one end of the same electrical charge to the next to the grounded collector (10). During the process, this polymer jet follows prior stable then unstable (spiral) track path. During this time, the solvent in it evaporates and leaves behind a charged polymeric fiber having diameters in the nano scale. The resulting continuous nanofibers are randomly positioned on the collector plate (10) and form a nonwoven mat [61-63].

Electrospinning is an inexpensive and simple method of producing nanofibers, while controllability is a very difficult process. Because there are many technical parameters affecting the process [9]. The electrospinning process and the structure and morphology of the nanofibers obtained from this process are directly related to the parameters collected in the three main headings as solution properties, process conditions and ambient conditions. The properties of the polymer solution are the most important parameters affecting the electrospinning process and the morphology of the formed fiber. Surface tension plays an important role in the formation of beads, one of the most common problems on nanofibrous mats. The solution viscosity and electrical properties are influential on the formation and movement of the polymer jet. All have an effect on the diameter of electrospinning fibers [64]. Another important parameter that affects the electrospinning process is the various external factors that influence the electrospinning jet. These factors include the voltage applied, the feed rate, the solution temperature, the collector type, the nozzle diameter and the distance between the nozzle and the collector. Although not as much as the solution parameters, process parameters also have a significant effect on the resulting fiber diameter and morphology [65, 66].

During the electrospinning process, when the polymer jet is separated from the Taylor cone (20) formed at the tip end, the polymer jet is extended and stretched by the influence of the electrostatic forces, the Coulomb repulsion forces, etc. as they travel towards the collector (10). In the process of stretching the solution, it is the complexity of the molecular chains that prevent breaks in the electrically moving polymer jet and thus allows the formation of a continuous solution jet. The increase in solution viscosity causes the polymer chain complexity to increase and overcomes the surface tension forces, thereby ensuring jet continuity during the electrospinning process. With an increase in viscosity, the bead shape changes from spherical to spindle-like structure while the formation of beads in the nanofibrous mat decreases. With increased viscosity, fiber diameters also increase due to the decrease of the jet path. However, too high viscosity will make it difficult to pump the solution from the nozzle. In very low viscosities, bead formation occurs along the nanofibers, there is low chain complexity, and the surface tension forces on the polymer jet are dominant. Electrospray occurs instead of electrospinning, and polymer particles are formed on the mat instead of fibers [54, 63, 65, 67].

The electrically charged solution must come from above the surface tension so that the electrospinning can begin. That is, surface tension is a factor that makes electrospinning difficult. Depending on the surface tension, when the concentration of free solvent molecules is high, the tendency of the solvent molecules to aggregate and take up a global shape will increase. In this case, surface tension may cause beads to form along the jet as the polymer jet travels toward reservoir plate (10). High viscosity means more interaction between solvent and polymer molecules, so that when the solution is stretched by the action of the charge, the solvent molecules will diffuse into the complex polymer molecules, thereby reducing the tendency of the solvent molecules to aggregate under the influence of surface tension [63-65].

In the electrospinning process, the polymer jet is stretched by pushing the loads on the surface together. If the electrical conductivity of the solution is increased, more charge may be carried in the electrospinning jet. If the solution is not fully stretched, pilling will occur. Another effect of the increased load is to increase the collection area of the fibers to produce finer fibers. There is a correlation between the pH and the conductivity of the solution. The negatively charged OH ions present in a solution prepared in basic conditions have a significant effect on the increase of electric conductivity and jet tension. Although the electrical conductivity is advantageous for the electrospinning process, it has a decisive effect, which makes the process more difficult, even after a certain limit. At very high conductivity values, it is very difficult to maintain the load on the droplet surface at the tip of the needle in electrospinning, and this affects the formation of the characteristic cone (20). As conductivity increases, the classical cone-jet pattern changes and multijet formation can be seen [68, 69].

The applied high voltage ensures that the polymer solution with certain electrical conductivity is electrically charged and the electrostatic forces which cause the solution to travel in a thin jet towards a grounded collector (10). Electrospinning processes start when the electrostatic forces acting on the resistors absorb the surface tension forces of the solution. When a voltage is applied, the resulting electric field affects the jet tension and acceleration. That is, both the voltage and the electric field obtained have an influence on the morphology of the fiber obtained. When a higher voltage is applied, due to the higher Coulomb power in the jet and the stronger electric field, the solution will stretch further. As this situation leads to a reduction in fiber diameter at the same time, it causes the solvent to evaporate more rapidly, resulting in more dry fibers [65, 70].

The feed rate defines the amount of solution available for electrospinning. To keep the Taylor cone (20) stable, there is a corresponding feed rate for a given voltage. As the feed rate increases, the volume of the solution coming from the gland increases, resulting in an increase in fiber diameter or bead size. Due to the greater volume of solution coming out of the nozzle, the drying of the jet takes longer. As a result, the solvent in the fibers collected during the same flight does not have enough time to evaporate. Some solvent remaining after evaporation will evaporate after the fibers are placed on the collector (10), so that sticking can occur at points where the fibers are in contact with each other [65, 66].

The distance between the collector (10) and the nozzle is the area where the jetting occurs, the jet is incinerated, and the solvent evaporates and forms solid fibers. That is, the electrospinning process takes place at this distance. The flight time and the electric field strength of the polymer jet in the air until reaching the collector (10) are factors affecting the electrospinning process and formed fibers. By changing the distance between the nozzle and the collector (10), both the flight time and the electric field strength are changed. When the distance is shortened, this time will be shortened and since the solvent does not evaporate completely, the sticks and bead formations will be seen at the contact points of the fibers. When the distance is increased, the electric field strength will increase and the jet speed will increase and the fiber diameters will decrease [65, 67].

HA and NaAlg used to form the nanofibrous mat subject of the invention are water-soluble polymers. The resulting nanofibrous mats have low resistance to water and water vapor. This would lead to problems in practical applications of nanofibrous mats that are planned to be used as adhesion barriers it is necessary to make an appropriate cross-linking treatment on the produced mats to increase the water resistance. Cross-linking is the process of attaching two or more molecules together by covalent bonding with a chemical method. Cross-linking of the adhesion zone nanofibrous mat is carried out in the presence of 1-ethyl-3-(3-dimethylaminopropyl) carbodiimide hydrochloride (EDC) and N-hydroxysuccinimide (NHS).

EDC is a water-soluble, biocompatible and non-toxic cross-linking agent used to couple carboxyl groups to primary amines. The non-inclusion of EDC in the cross-linked structure, i.e., not binding to polymer molecules, is particularly recommended for materials used in the biomedical field [50, 51]. The chemical structure of EDC is given below.

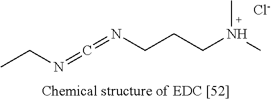

EDC binds by activating carboxyl groups on polysaccharide molecules and forming ester bonds between hydroxyl and carboxyl groups. In the reaction mechanism exemplified below, the carboxyl groups of the HA polymer are activated with carbodiimide and form an O-acylurea labile intermediate product. This unstable intermediate product is short-lived and breaks down to link the hydroxyl and carboxyl groups of the HA polymer [53, 54].

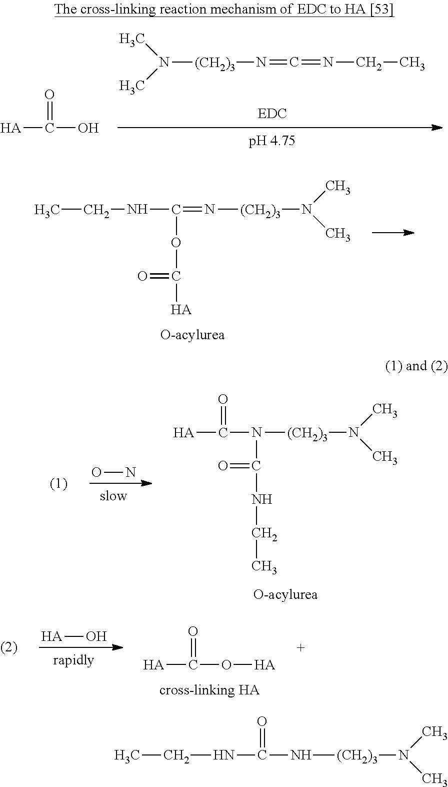

NHS is a cross-linking agent used to activate carboxylic acid groups. When a normal carboxylic acid forms salt with amines, the acids which are activated in the presence of NHS react with amines to give amides [54]. The chemical structure of NHS is given below.

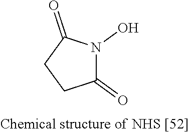

EDC productivity increases in NHS presence. NHS is a homo-bifunctional crosslinker. This cross-linker is used in conjunction with EDC to inhibit the formation of irreversible N-acylic compounds. The combined use of EDC and NHS provides intermediate formulations that are hydrolytically resistant and cannot be regenerated. NHS has no toxic properties and imparts properties such as biocompatibility to the material, resistance to enzymatic degradation. NHS provides cross-linking by activating the esters of the glucuronic acid moiety present in the HA polymer [55]. Below, the EDC/NHS cross-linking reaction mechanism of HA is given.

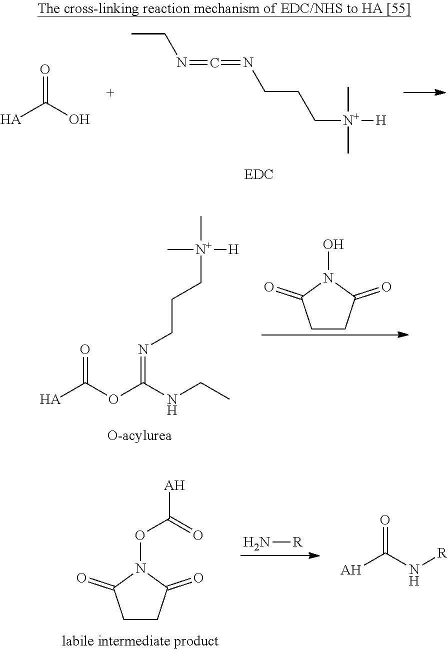

In general, the method of producing the nanofibrous mat of the present invention comprises;
The preparation of hyaluronic acid solution in NaOH/Dimethyl sulfoxide (DMSO)
The preparation of aqueous sodium alginate solution,
The mixing of the two solutions prepared,
The application of the electrospinning method to the mixture solution,
The cross-linking of the obtained nanofibrous mat.

### Nanofibrous Mat Production

First, 2% aqueous NaAlg and 12% HA (in NaOH/DMSO) solutions were prepared. The measured properties of the prepared solutions are given in Table 2-3.

TABLE 2
Properties of the aqueous NaAlg polymer solution

| | | Electrical | | Surface |
|---|---|---|---|---|
| | | Conductivity | Viscosity | Tension |
| Concentration | pH | (μS/cm) | (cP) | (mN/m) |
| 2% | 7.36 | 6600 | 4915 | 65.97 |

TABLE 3
Properties of NaOH/DMSO/HA polymer solutions

| | | Electrical | | Surface |
|---|---|---|---|---|
| | | Conductivity | Viscosity | Tension |
| Concentration | pH | (μS/cm) | (cP) | (mN/m) |
| 12% | 14 | 67800 | 2816 | 29.67 |

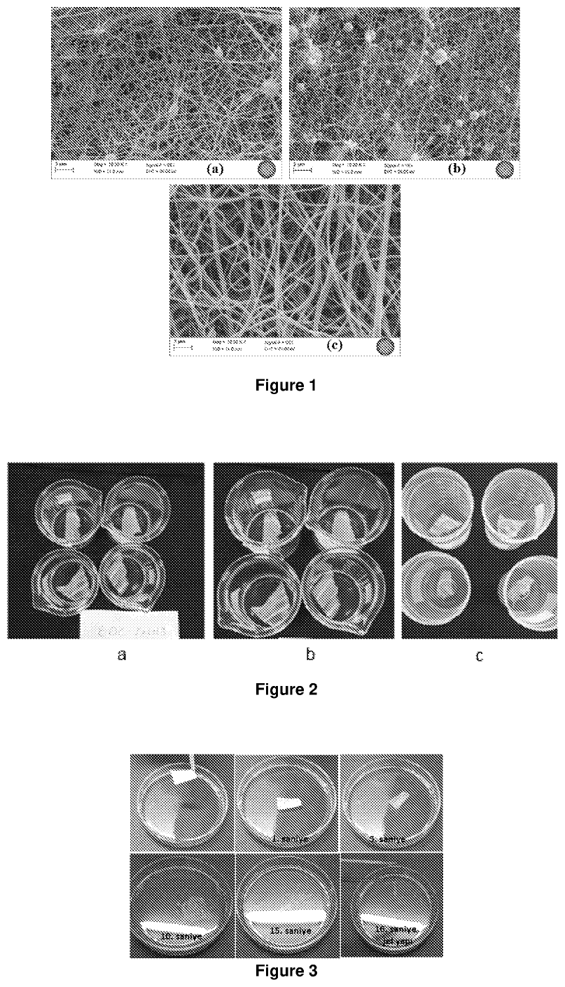

Subsequently, mixtures of 12% HA/2% NaAlg solutions were prepared at ratios of 2/1, 3/1 and 5/1. Nanofibrous mat was produced by electrospinning. Solution properties are shown in Table 4, and Scanning Electron Microscopy (SEM) views are given in FIG. 1.

TABLE 4
HA/NaAlg polymer blend solution and production parameters

| Polymer 1 (P1) | HA | HA | HA |
|---|---|---|---|
| Polymer 2 (P2) | NaAlg | NaAlg | NaAlg |
| Solvent1 for P1 (Ç1) | NaOH | NaOH | NaOH |
| Solvent2 for P1 (Ç2) | DMSO | DMSO | DMSO |
| Solvent for P2 | Pure water | Pure water | Pure water |
| Ç1/Ç2 mixing ratio (v/v) | 4/1 | 4/1 | 4/1 |
| P1 concentration (w/v %) | 12 | 12 | 12 |
| P2 concentration (w/w %) | 2 | 2 | 2 |
| P1/P2 mixing ratio (v/v) | 2/1 | 3/1 | 5/1 |
| pH | 12.91 | 12.97 | 12.95 |
| Conductivity (μS/cm) | 55620 | 48760 | 47260 |
| Viscosity (cP) | 995.2 | 2438 | 713.6 |
| Surface tension (mN/m) | 28.83 | 23.30 | 26.52 |
| Distance (cm) | 9.5 | 8 | 7.5 |
| Voltage (kV) | 19 | 20 | 18.7 |
| Feed amount (ml/h) | 0.25 | 0.4 | 0.6 |
| Average nanofiber diameter (nm) | 130 ± 23 | 76 ± 19 | 108 ± 57 |

### Cross-Linking Process

50, 70, 80, and 100 mM EDC and 100 mM NHS to provide cross-linking were dissolved in ethanol. Into four EDC/NHS mixture solutions prepared in a volume ratio of 1/1, nanofibrous mat samples were immersed and allowed to stand at room temperature for 24 hours. After this time, the nanofiber cross-linked without any structural degradation, gelling or dissolution were removed from the solutions, rinsed in ethanol and allowed to dry for 12 hours at 37° C. in the incubator. FIG. 2 shows the cross-linking images made with EDC/NHS.

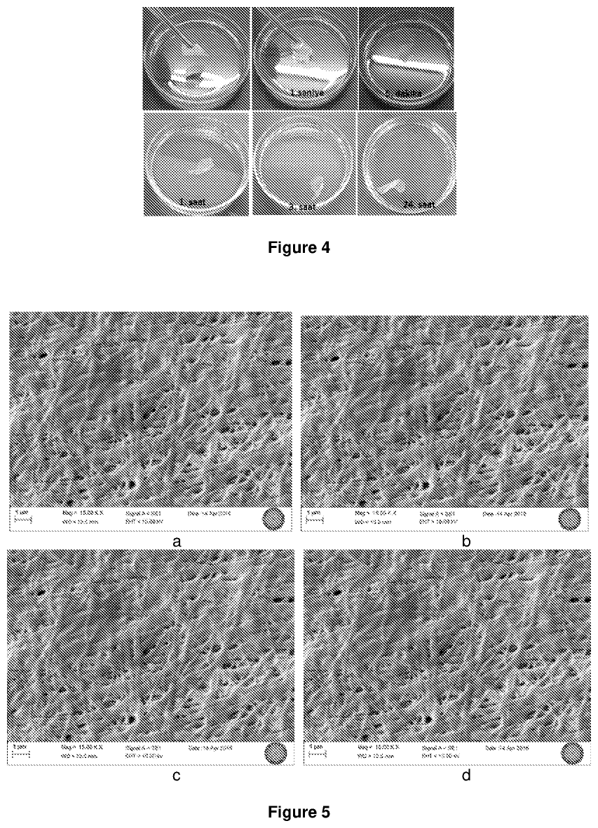

Following the drying process, in order to understand whether the cross-linking agent used improves the water resistance of the mats, and the mats that cross-linked and had not been cross-linked were left to stand in purified water at room temperature for 24 hours. It is seen that the non-crosslinked mat is rapidly dispersed and entered into the gelling process from the first seconds after it was thrown into the water. In the 15th second following the moment when it was thrown in the water, the mat is completely gelated. In FIG. 3, the water resistance test of the nanofibrous mat is given before cross-linking. After the cross-linking methods employed, it has been observed that the cross-linked mats do not dissolve in water and maintain their dimensional stability. FIG. 4 shows the water resistance test of the nanofibrous mat after cross-linking with 80 mM/100 mM EDC/NHS cross-linking agent. Furthermore, it is understood that the fibrous structure is maintained in the mat after crosslinking SEM images in FIG. 5. In the production of the nanofibrous mat subject of the invention; it is possible to use;
Instead of the HA/NaAlg mixture solution, the HA/CMC/NaAlg mixture solution,
Dimethyl formamide (DMF) instead of DMSO,
Instead of ethanol; methanol,
Instead of NHS, divinyl sulfone (DVS).

In the production of the nanofibrous mat subject of the invention; it is possible that the polymer, the polymer mixture solution and the production parameters are within the following ranges.
HA concentration to be prepared in NaOH/DMSO; 8-15% (w/v %)
NaAlg concentration to be prepared in pure water; 1-4% (w/w %)
The mixing ratio of the two polymer solutions (HA/NaAlg); 1/1-2/1-3/1-4/1-5/1-6/1-7/1-8/1-9/1-10/1 (v/v)
Distance between the nozzle and the collector during electrospinning of solution; 7-15 cm
Voltage to be applied during the electrospinning of the solution; 15-25 kV
The feed rate of the solution during electrospinning; 0.2-0.8 ml/h
The amount of EDC to dissolve in ethanol during cross-linking; 50-100 mM
The amount of NHS to dissolve in ethanol during cross-linking; 50-100 mM
EDC/NHS mixture ratio during cross-linking; 1/1-3/1

A lot of material was used to prevent adhesion formation until today, but it has not been precisely shown that no one blocks the intra-abdominal adhesion. Studies continue to reduce or prevent intra-abdominal adhesions with used materials and with the products put forward constitute the million dollar health market.

The invention includes a low cost nanofibrous product that can reduce postoperative complications and has an alternative to commercial products used in the prior art, having a potency to inhibit/reduce adhesion by using electrospinning from sodium alginate and hyaluronic acid polymers during intra-abdominal surgical operations.

Different polymers have been used for the first time for nanofibrous mats recommended as an adhesion barrier. Some of these polymers (hyaluronic acid) are also polymers used in the production of commercial barriers. However, the polymer advantages of commercial barriers (gel, membrane, etc.) in different constructions are combined with the advantages of a nanofibrous mat.

The natural polymers HA and NaAlg are used in mixture with different polymers or purely as a gel, film, membrane or fiber/nanofiber biomedical field. Previously, however, no nanofibrous mat was produced by electrospinning from the HA/NaAlg polymer mixture. In addition, no nanofibrous mat has been produced from these polymers, either alone or in admixture, for use as an adhesion barrier.

Nanofibrous mats are the materials proven in tissue engineering due to its flexibility, large surface area, nano-porous structure, oxygen permeability, non-bacterial permeability, similarity to natural tissue, favorable cell growth and be inexpensive of production.

According to the invention, the adhesion barrier formed of a nanofibrous mat is an easier, cheaper and more effective alternative to commercial adhesion barriers used in the market.

The insertion of the adhesion barriers into the tissue is carried out as follows.

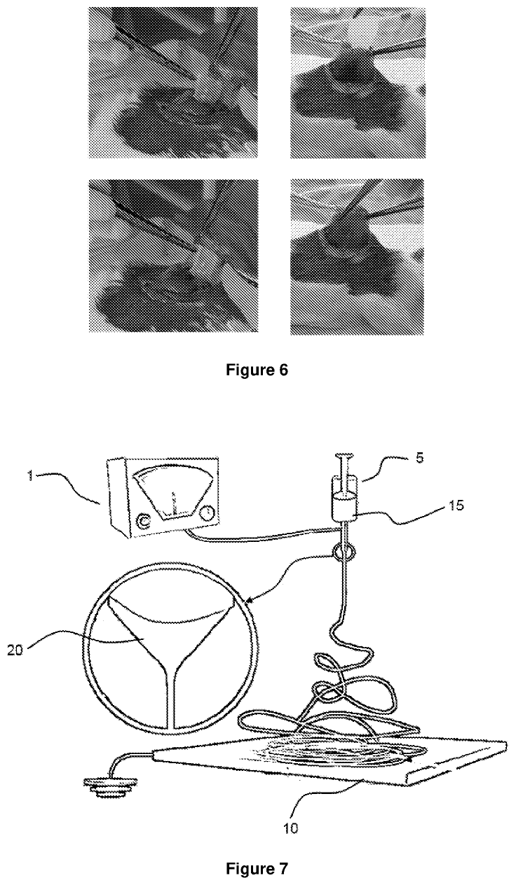

In practice, the adhesion barrier is placed by intraperitoneal onlay technique between the peritoneum and intra-abdominal organs or between the rectus posterior sheath and the peritoneum. During the application of adhesion barrier, suturing is not necessary due to the gelling property of the material. Produced adhesion barrier brings an advantage, as the substance and type for the suturation during the operation does not cause any foreign body reactions. The barrier placement process with intra-peritoneal onlay technique is schematically shown in FIG. 6.

Thanks to the invention, the following advantages have been achieved according to the adhesion barrier in the patent of TR 2016 10544, which belongs to the right owners named ESRA KARACA, ŞERIFE ŞAFAK, and RABIA GÖZDE ÖZALP.

### 1—According to the Biodegradability Test Results:

During the biodegradability test, the samples of HA/CMC/NaAlg and HA/NaAlg nanofibrous mats were incubated in phosphate buffered saline (DPBS) for 12 hours, 1; 1.5; 2; 3; 5 and 7 days at 37° C. The biodegradation ratios were determined by measuring weight loss on the mats after the end of the specified periods. The calculated values were given in Table 5.

TABLE 5
Biodegradability test results of the nanofibrous mats

| | Weight loss (%) | | | | | | |
|---|---|---|---|---|---|---|---|
| Nanofibrous | 12 | 1 | 1.5 | 2 | 3 | 5 | 7 |
| mat | hours | day | days | days | days | days | days |
| HA/NaAlg | 6.02 | 9.85 | 12.79 | 21.27 | 26.03 | 47.64 | 75.06 |
| HA/CMC/NaAlg | 7.84 | 10.78 | 16.92 | 22.72 | 26.70 | 41.07 | 71.72 |

Both of the mats showed the weight loss in a linearly increasing manner for seven days. The critical period for adhesion formation is the first seven days after trauma. The mechanism of adhesion formation follows a very rapid course in this period. For this reason, an ideal surgical adhesion barrier should be able to maintain its presence by keeping the tissues separated from each other for the first seven days [56]. According to the biodegradation test results, the nanofibrous mats could protect their structures sufficiently during the critical healing period. However, in the first five days, HA/NaAlg nanofibrous mat was degraded more slowly than the HA/CMC/NaAlg nanofiber mat.

### 2—According to the Cytotoxicity Test Results Under In Vitro Conditions:

The nanofibrous mat aimed as an adhesion barrier must not have any toxic effects, and it should not affect wound healing in the abdominal region. Therefore, possible cytotoxic effects of the nanofibrous mats were investigated by using human umbilical vein/vascular endothelial cell line (HUVEC) and mouse subcutaneous connective tissue fibroblast cell line (L-929) by the XTT cell viability test according to ISO 10993-5:2010. The cell viability values obtained after 24 hours were given in Table 6.

TABLE 6
Cytotoxicity test results of the nanofibrous mats

| | HUVEC cell viability (%) | | L929 cell viability (%) | |
|---|---|---|---|---|
| | | Standard | | Standard |
| Nanofibrous mat | Mean | Deviation | Mean | Deviation |
| HA/NaAlg | 95.45 | 11.00 | 95.94 | 13.00 |
| HA/CMC/NaAlg | 91.38 | 2.80 | 92.54 | 11.00 |

According to the standard, samples are considered as non-cytotoxic if the cell viability is above 70%. When the cytotoxicity results examined, no cytotoxic effects of both nanofibrous mats were observed. However, the percentage cell viability was detected higher in HA/NaAlg nanofibrous mat.

### 3—According to the Adhesion Scoring Results Under In Vivo Conditions:

Performance of the nanofibrous mats as an adhesion barrier was evaluated by using eight Wistar-Albino female rats in each group, in weighing 250-300 g. After the disinfection of the abdominal region with Baticon solution, abdominal cavity was opened by skin incision in 2 cm on the ventral abdomen. A median laparotomy was carried out and the musculo-peritoneal defect was formed by resection of a segment of the rectus abdominis. In addition, the cecum was taken out, and serosal petechiae were formed on the cecum by rubbing 15 times with sterile gauze. In applications, the nanofibrous adhesion barrier was placed between the defected muscle and injured cecum.

Intra-abdominal adhesions in the rats were evaluated macroscopically according to the Modified Diamond Scale (Table 7). The results of adhesion scoring of the nanofibrous mats were given in Tables 8 and 9.

TABLE 7
Adhesion grading scores according
to Modifiye Diamond Scale [57]

| | Tenacity of | Appearance and | Extent of |
|---|---|---|---|
| Score | adhesion | severity of adhesion | adhesion |
| 0 | No adhesion | No adhesion | 0 |
| 1 | Easy dissection | Thin, filmy transparent | <25% |
| | | avascular | |
| 2 | Moderate dissection | Opaque, translucent | 25-50% |
| | | avascular | |
| 3 | Blunt dissection | Opaque, translucent, | 50-75% |
| | | Limited vascular | |
| 4 | Sharp dissection | Opaque, well- | >75% |
| | | vasculated | |

TABLE 8
Individual adhesion scores of the rats and mean adhesion
scores of the group for HA/NaAlg nanofibrous mat

| Experimental Animal | 1 | 2 | 3 | 4 | 5 | 6 | 7 | 8 |
|---|---|---|---|---|---|---|---|---|
| Adhesion score | 0 | 0 | 1 | 0 | 1 | 0 | 0 | 0 |

| | |
|---|---|
| Mean | 0.25 |

| Experimental Animal | 1 | 2 | 3 | 4 | 5 | 6 | 7 | 8 |
|---|---|---|---|---|---|---|---|---|
| Adhesion score | 2 | 1 | 4 | 1 | 2 | 2 | 1 | 1 |

| | |
|---|---|
| Mean | 1.75 |

As a result of the macroscopic evaluation; it was determined that HA/NaAlg nanofibrous mat prevented the adhesion formation more effectively than HA/CMC/NaAlg nanofibrous mat in the abdominal region of the rats. The statistical difference between the adhesion scoring of both mats was found (p=0.002).

4—According to the Histopathological Evaluation Results under in Vivo Conditions:

After the macroscopic adhesion scoring, the tissue samples from different regions were subjected to histopathological evaluation. The sections stained with Hematoxylin-Eosin and Mason Trichrome were investigated regarding inflammation, fibrosis, neovascularization and increase of the collagen fibrils in light microscopy, respectively.

When an injury occurs in the peritoneum, fibrin gel matrix forms for the healing. The fibrin gel matrix starts to form adhesion by coagulating in the first hours. Initially, most of the fibrin gel matrix is absorbed by degrading with fibrinolytic activity in the peritoneum. If the fibrinolytic activity is insufficient and the resulting adhesion remains for three days or more, fibroblastic proliferation develops within the fibrin gel matrix and becomes permanent adhesions (fibrosis tissues). The inflammation in damaged tissues is another factor that triggers the formation of the fibrin gel matrix [3-5]. Also, it is pointed out that the formation of neovascularization is also observed together with the adhesion [1]. So, the formation of inflammation, fibrosis, and neovascularization is considered to be the presence of adhesions and triggers the formation of adhesion during the postoperative healing. Therefore, the adhesion in the rat groups was assessed by scoring the formations of inflammation (Table 10), fibrosis (Table 11), and neovascularization (Table 12).

TABLE 10
Inflammation grading scale [58]

| Score | Assessment |
|---|---|
| 0 | Nil |
| 1 | Giant cells, occasional scattered lymphocytes and |
| | plasma cells |
| 2 | Giant cells with increased numbers of admixed |
| | lymphocytes, plasma cells, eosinophils, neutrophils |
| 3 | Many admixed inflammatory cells, microabscesses present |

TABLE 11
Fibrosis grading scale [58]

| Score | Assessment |
|---|---|
| 0 | Nil |
| 1 | Minimal, loose |
| 2 | Moderate |
| 3 | Florid, dense |

TABLE 12
Neovascularization grading score [1]

| Score | Inflammatory cell/Fibroblast/Neovascularization/Collagen |
|---|---|
| 0 | No evidence |
| 1 | A small amount and scattered |
| 2 | A small amount and all areas |
| 3 | There are a lot and scattered |
| 4 | There are a lot and all areas |

### Inflammation

The distributions of inflammation scores of the nanofibrous mats according to the groups were shown in Table 13.

TABLE 13
Distribution of inflammation scores according to the groups

| Inflammation score | HA/CMC/NaAlg | HA/NaAlg | | Total |
|---|---|---|---|---|
| 0 | — | — | | 0 |
| 1 | — | 5 | (62.5%) | 5 |
| 2 | 7 (87.5%) | 2 | (25%) | 9 |
| 3 | 1 (12.5%) | 1 | (12.5%) | 2 |

| | | | |
|---|---|---|---|
| Total | 8 | 8 | 16 |

In the group of rats used HA/CMC/NaAlg nanofibrous mat was met inflammation score 2 mostly, whereas the rats used HA/NaAlg nanofibrous had shown inflammation score 1 mostly. So, it was understood that HA/NaAlg nanofibrous mat prevented the inflammation more effectively than HA/CMC/NaAlg nanofibrous mat. The statistically significant difference (p=0.026) was also found between the inflammation scores of both nanofibrous mats.

### Fibrosis

The distributions of fibrosis scores of the nanofibrous mats according to the groups were shown in Table 14.

TABLE 14
Distribution of fibrosis scores according to the groups

| Fibrosis score | HA/CMC/NaAlg | HA/NaAlg | | Total |
|---|---|---|---|---|
| 0 | — | 1 | (12.5%) | 1 |
| 1 | — | 6 | (75%) | 6 |
| 2 | 2 (25%) | 1 | (12.5%) | 3 |

| | | | |
|---|---|---|---|
| 3 | 6 (75%) | — | 6 |
| Total | 8 | 8 | 16 |

In the group of rats used HA/CMC/NaAlg nanofibrous mat was met fibrosis score 3 mostly, whereas the rats used HA/NaAlg nanofibrous had shown fibrosis score 1 mostly. So it was understood that HA/NaAlg nanofibrous mat prevented the fibrosis more effectively than HA/CMC/NaAlg nanofibrous mat. There was a statistically significant difference (p=0.001) between the fibrosis scores of both nanofibrous mats.

### Neovascularization

The distributions of neovascularization scores of the nanofibrous mats according to the groups were shown in Table 15.

TABLE 15
Distribution of neovascularization scores according to the groups

| Neovascularization score | HA/CMC/NaAlg | HA/NaAlg | Total |
|---|---|---|---|
| 0 | — | 3 (37.5%) | 3 |
| 1 | 7 (87.5%) | 5 (62.5%) | 12 |
| 2 | 1 (12.5%) | — | 1 |
| 3 | — | — | 0 |
| Total | 8 | 8 | 16 |

In the groups of rats used both HA/CMC/NaAlg and HA/NaAlg nanofibrous mats were met neovascularization score 1 mostly. However, no neovascularization was detected in 3 groups, in which HA/NaAlg nanofibrous mat was used. It was found that there was no statistical difference (p=0.511) between the groups for the neovascularization score of both nanofibrous mats.

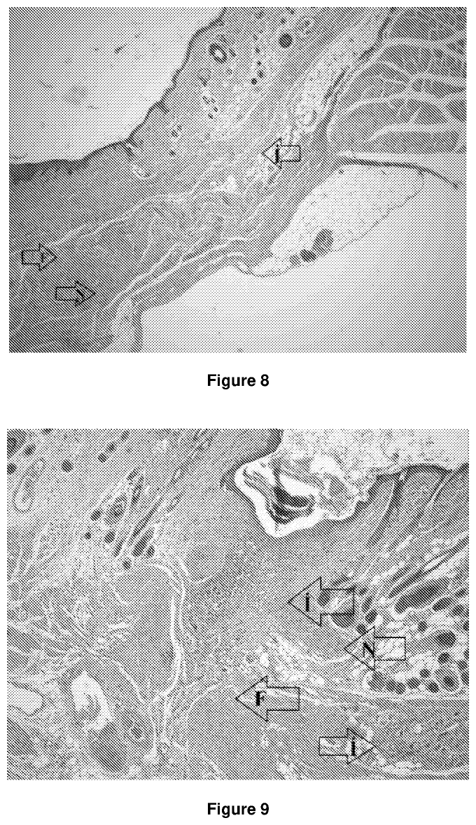

Also, the exemplary views of fibrosis, inflammation, and neovascularization on sections taken from the defected sites in rat groups were given in FIGS. 8 and 9.

Under the view of these results; it has been observed that HA/NaAlg nanofibrous mat significantly reduced the formation of inflammation, fibrosis, and neovascularization compared to other nanofibrous mat.

### Collagen Fibrillation

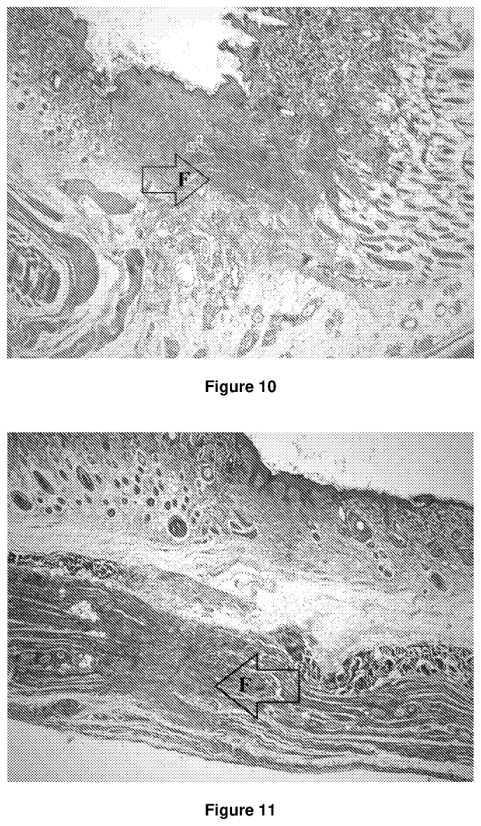

In the formation of adhesions; the onset of collagen fibril formation is after the fifth day of injury or trauma in the tissue. If the adhesion formation cannot be prevented because of lack of fibrinolytic activity, collagen accumulation continues in the tissue. Collagen fibrils organize in one month and cause maturation of the adhesion into fibrosis band structure [3-5]. Collagen fibrils formed in tissues can be easily distinguished by Mason Trichrome (MTK) histochemical staining. The sections obtained from the defected regions of the rat groups were stained with MTK and evaluated under the light microscope for the formation of fibrosis due to collagen fibril. The images were shown in FIGS. 10 and 11.

In the photos of HA/CMC/NaAlg nanofibrous mat; collagen fibrils (blue color), which are more distinct and more severe, disrupted the integrity of the tissue by entering between the muscle tissue (red color) and caused the adhesion. On the other hand, mild adhesion was observed for HA/NaAlg nanofibrous mat. As a result, it was determined that HA/Nalg nanofibrous mat decreased the adhesion more effectively than HA/CMC/NaAlg nanofibrous mat.

### REFERENCE LIST

1. Sümer, A. 2005. Effects of Oxidized Regenerated Cellulose, Polyethylene Glycol and Hylan Gf-20 on Adhesions after Prolene Mesh Application. Specialization Thesis, Ministry of Health Haydarpaşa Numune Training and Research Hospital, Istanbul.
2. Akçil, A. M. 2008. Creation of Tissue Barrier with Sodium Hyaluronate and Carboxymethylcellulose Membrane (Seprafilm®) to Prevent Adhesion After Surgery in Rabbit Mediastinum. Specialization Thesis, Istanbul University, Istanbul.
3. Şahiner, I. T. 2011. Comparison of Intraabdominal Adhesions in the Repair of Abdominal Wall Defects with Simvastatin Loaded Polypropylene Patch. Specialization Thesis, Kirikkale University, Kirikkale.
4. Ye\check{g}eno\check{g}lu, A. 2006. Comparison of the Activities of Heparin, Seprafilm, Heparin and Seprafilm in the Prevention of Postoperative Intraperitoneal Adhesions. Specialization Thesis, Ministry of Health Dr. Lütfi Kirdar Training and Research Hospital, Istanbul.
5. Altinel, Y. 2011. Comparison of Two Different Polypropylene Mesh and Chitin Coated Forms in the Treatment of Abdominal Wall Hernia in Rats. Specialization Thesis, Uluda\check{g} University, Bursa.
6. Ensari, N. 2008. Its Application to the Middle Ear in Order to Investigate the Neurotoxic Effect of Polylactic Acid Bioabsorbable Adhesion Barrier Film in Guinea Pig Specialization Thesis, Gazi University, Ankara.
7. Günaydin, M., Güvenç, D., Yildiz, L., Aksoy, A., Tander, B., Biçakci, Ü., Ayyildiz, H. S., Sünter, A. T., Bernay, F. 2012. “Comparison of the Materials Used to Prevent Abdominal Adhesion: An Experimental Study in Rats”, Turkish Clinics Journal of Medical Sciences, 32(2), 337-345.
8. Malçik, H. 2009. The Effect of Proanthocyanidine on the Development of Experimental Intraabdominal Adhesions in Rats. Specialization Thesis, Ministry of Health Taksim Training and Research Hospital, Istanbul.
9. Cengiz, F., Krucinska, I., Gliscinska, E., Chrzanowski, M., Göktepe, F. 2009. “Comparative Analysis of Various Electrospinning Methods of Nanofibre Formation”, Fibres & Textiles in Eastern Europe, (17) 13-19.
10. Jian, F., HaiTao, N., Tong, L., XunGai, W. 2008. “Applications of Electrospun Nanofibers”, Chinese Science Bulletin, 15, 2265-2286.
11. Huang, C., Chen, S., Lai, C., Reneker, D. H., Qiu, H., Ye, Y., Hou, H. 2006. “Electrospun Polymer Nanofibres With Small Diameters”, Nanotechnology, 17, 1558-1563.
12. Kriegel, C., Arrechi, A., Kit, K, Mcclements, D. J, Weiss, J. 2008, “Fabrication, Functionalization, and Application of Electrospun Biopolymer Nanofibers”, Critical Reviews in Food Science and Nutrition, 48(8), 775-797.
13. Kumar, A. 2010. Nanofibers. Croatia: Intech.
14. Patanaik, A., Anandjiwala, R. D, Rengasamy, R. S, Ghosh, A, Pal, H. 2007. Nanotechnology in Fibrous Materials—A New Perspective. London: Taylor & Francis.
15. Huang, Z. M., Zhang, Y. Z., Kotaki, M., Ramakrishna, S. 2003. “A review on Polymer Nanofibers by Electrospinning and their Applications in Nanocomposites”, Composites Science and Technology, 63(15), 2223-2253.
16. Üstünda\check{g}, C. G., Karaca, E., Özbek, S., Çavuşo\check{g}lu, I., 2010. “In Vivo Evaluation of Electrospun Poly (Vinyl Alcohol)/Sodium Alginate Nanofibrous Mat as Wound Dressing”, Tekstil ve Konfeksiyon, 20, 290-298.
17. Zhong, S. P., Zhang, Y. Z., Lim, C. T., 2010. “Tissue Scaffolds for Skin Wound Healing and Dermal Reconstruction”, WIREs Nanomed Nanobiotechnology, 2, 510-525.
18. Yoo, H. S., Kim, T. G., Park, T. G. 2009. “Surface-Functionalized Electrospun Nanofibers for Tissue Engineering and Drug Delivery”, Advanced Drug Delivery Reviews, 61(12), 1033-1042.
19. Kenawy, E. R., Abdel-Hay, F. I., El-Newehy, M. H., Wnek, G. E. 2009. “Processing of Polymer Nanofibers Through Electrospinning as Drug Delivery Systems”, Materials Chemistry and Physics, 113(1), 296-302.
20. Zeng, J., Xu, X., Chen, X., Liang, Q., Bian, X., Yang, L., Jing, X. 2003. “Biodegradable Electrospun Fibers for Drug Delivery”, Journal of Controlled Release, 92(3), 227-231.
21. Li, C., Vepari, C., Jin, H. J., Kim, H. J., Kaplan, D. L. 2006. “Electrospun Silk-BMP-2 Scaffolds for Bone Tissue Engineering”, Biomaterials, 27, 3115-3124.
22. Min, B. M., Lee, G. N., Kim, S. H., Nam, Y. S., Lee, T. S., Park, W. H. 2004. “Electrospinning of Silk Fibroin Nanofibers and Its Effect on the Adhesion and Spreading of Normal Human Keratinocytes and Fibroblasts in vitro”, Biomaterials, 25, 1289-1297.
23. VanugopaL, J. R., Zhang, Y., Ramakrishna, S. 2006. “In Vitro Culture of Human Dermal Fibroblasts on Electrospun Polycaprolactone Collagen Nanofibrous Membrane”, Artif Organs 30, 440-446.
24. Zhang, X., Reagan, R. M., Kaplan D. L. 2009. “Electrospun Silk Biomaterial Scaffolds for Regenerative Medicine”, Advanced Drug Delivery Reviews, 61, 988-1006
25. Diraço\check{g}lu D. 2007. “Intraarticular Hyaluronic Acid Treatment in Osteoarthritis-Education”, Turkish Journal of Physical Medicine and Rehabilitation, 53, 154-159.
26. Aytar, P., Buruk, Y., Çabuk, A. 2013. “Determination of Optimum Conditions of Hyaluronic Acid Production with Streptococcus equi by Plackett-Burman Method”, Electronic Microbiology Review, 11(1) 28-35.
27. Wang, X., Um, I. C., Fang, D., Okamoto, A., Hsiao, B. S., Chu, B. 2005. “Formation of Water-Resistant Hyaluronic Acid Nanofibers by Blowing-Assisted Electro-Spinning and Non-Toxic Post Treatments”, Polymer, 46(13), 4853-4867.
28. Ji, Y., Ghosh, K., Shu, X. Z., Li, B., Sokolov, J. C., Prestwich, G. D., Rafailovich, M. H. 2006. “Electrospun Three-Dimensional Hyaluronic Acid Nanofibrous Scaffolds”, Biomaterials, 27(20), 3782-3792.
29. Schante, C. E., Zuber, G., Herlin, C., Vandamme, T. F. 2011. “Chemical Modifications of Hyaluronic Acid for the Synthesis Of Derivatives for a Broad Range of Biomedical Applications”, Carbohydrate Polymers, 85(3), 469-489.
30. Collins, M. N., Birkinshaw, C. 2013. “Hyaluronic Acid Based Scaffolds for Tissue Engineering-A Review”, Carbohydrate Polymers, 92(2), 1262-1279.
31. Price, R. D., Berry, M. G., Navsaria, H. A. 2007. “Hyaluronic Acid: The Scientific and Clinical Evidence”, Journal of Plastic, Reconstructive & Aesthetic Surgery, 60(10), 1110-1119.
32. Li, J., He, A., Han, C. C., Fang, D., Hsiao, B. S., Chu, B. 2006. “Electrospinning of Hyaluronic Acid (HA) and HA/Gelatin Blends”, Macromolecular Rapid Communications, 27(2), 114-120
33. Li, J., He, A., Zheng, J., Han, C. C. 2006. “Gelatin and Gelatin-Hyaluronic Acid Nanofibrous Membranes Produced by Electrospinning of their Aqueous Solutions”, Biomacromolecules, 7(7), 2243-2247.
34. Liu, Y., Ma, G., Fang, D., Xu, J., Zhang, H., Nie, J. 2011. “Effects of Solution Properties and Electric Field on the Electrospinning of Hyaluronic Acid”, Carbohydrate Polymers, 83(2), 1011-1015.
35. Uppal, R., Ramaswamy, G. N., Arnold, C., Goodband, R., Wang, Y. 2011. “Hyaluronic Acid Nanofiber Wound Dressing-Production, Characterization, and in Vivo Behavior”, Journal of Biomedical Materials Research Part B: Applied Biomaterials, 97(1), 20-29.
36. Ji, Y., Ghosh, K., Li, B., Sokolov, J. C., Clark, R. A., Rafailovich, M. H. 2006. “Dual-Syringe Reactive Electrospinning of Cross-Linked Hyaluronic Acid Hydrogel Nanofibers for Tissue Engineering Applications”, Macromolecular Bioscience, 6(10), 811-817.
37. Kim, T. G., Chung, H. J., Park, T. G. 2008. “Macroporous and Nanofibrous Hyaluronic Acid/Collagen Hybrid Scaffold Fabricated by Concurrent Electrospinning and Deposition/Leaching of Salt Particles”, Acta Biomaterialia, 4(6), 1611-1619.
38. Yao, C., Li, X., Song, T. 2007. “Fabrication of Zein/Hyaluronic Acid Fibrous Membranes by Electrospinning”, Journal of Biomaterials Science, Polymer Edition, 18(6), 731-742.
39. Qin, Y. 2008. “Alginate Fibres: An Overview of the Production Processes and Applications in Wound Management”, Polymer International, 57, 171-180.
40. Seventekin, N. 2001. Kimyasal Lifler. Izmir: Ege Üniversitesi Tekstil ve Konfeksiyon Araştirma-Uygulama Merkezi Yayinlari.
41. http://www.fao.org/docrep/W6355E/w6355e0x.htm, 2014. Son Erişim Tarihi: 18 A\check{g}ustos 2014.
42. Üstünda\check{g}, C. G. 2009. Elektrospinning Yöntemi ile Biyomedikal Kullanima Yönelik Nanolif Yüzey Üretimi ve Uygulamasi. Yüksek Lisans Tezi, Uluda\check{g} Üniversitesi, Bursa.
43. Shalumon, K. T., Anulekha, K. H., Nair, S. V., Nair, S. V., Chennazhi, K. P., Jayakumar, R. 2011. “Sodium Alginate/Poly (vinyl alcohol)/Nano ZnO Composite Nanofibers for Antibacterial Wound Dressings”, International Journal of Biological Macromolecules, 49(3), 247-254.
44. Tarun, K., Gobi, N. 2012. “Calcium Alginate/PVA Blended Nano Fibre Matrix for Wound Dressing”, Indian Journal of Fibre & Textile Research, 37, 127-132.
45. Coşkun, G., Karaca, E., Ozyurtlu, M., Özbek, S., Yermezler, A., Çavuşo\check{g}lu, I. 2014. “Histological Evaluation of Wound Healing Performance of Electrospun Poly (Vinyl Alcohol)/Sodium Alginate as Wound Dressing in Vivo”, Bio-medical Materials and Engineering, 24(2), 1527-1536.
46. Jeong, S. I., Krebs, M. D., Bonino, C. A., Samorezov, J. E., Khan, S. A., Alsberg, E. 2010. “Electrospun Chitosan-Alginate Nanofibers With In Situ Polyelectrolyte Complexation for Use as Tissue Engineering Scaffolds”, Tissue Engineering Part A, 17(1-2), 59-70.
47. Jeong, S. I., Krebs, M. D., Bonino, C. A., Khan, S. A., Alsberg, E. 2010. “Electrospun Alginate Nanofibers with Controlled Cell Adhesion for Tissue Engineering”, Macromolecular Bioscience, 10(8), 934-943.
48. Ma, G., Fang, D., Liu, Y., Zhu, X., Nie, J. 2012. “Electrospun Sodium Alginate/Poly (Ethylene Oxide) Core-Shell Nanofibers Scaffolds Potential for Tissue Engineering Applications”, Carbohydrate Polymers, 87(1), 737-743.
49. Yang, D., Li, Y., Nie, J. 2007. “Preparation of Gelatin/PVA Nanofibers and Their Potential Application in Controlled Release of Drugs”, Carbohydrate Polymers, 69(3), 538-543.
50. Fischer, R. L., McCoy, M. G., Grant, S. A. 2012. “Electrospinning Collagen and Hyaluronic Acid Nanofiber Meshes”, Journal of Materials Science: Materials in Medicine, 23(7), 1645-1654.
51. Xu, S., Li, J., He, A., Liu, W., Jiang, X., Zheng, J., Han, C. C., Hsiao, B. S., Chu, B., Fang, D. 2009. “Chemical Crosslinking and Biophysical Properties of Electrospun Hyaluronic Acid Based Ultra-thin Fibrous Membranes”, Polymer, 50(15), 3762-3769.
52. https://www.thermofisher.com/order/catalog/product Son Erişim Tarihi: 12 Nisan 2016.
53. Lu, P. L., Lai, J. Y., Ma, D. H. K., Hsiue, G. H. 2008. “Carbodiimide Cross-linked Hyaluronic acid Hydrogels as Cell Sheet Delivery Vehicles: Characterization and Interaction with Corneal Endothelial Cells”, Journal of Biomaterials Science: Polymer Edition, 19(1), 1-18.
54. Tomihata, K., Ikada, Y. 1997. “Crosslinking of Hyaluronic Acid with Water-soluble Carbodiimide”, Journal of Biomedical Materials Research, 37(2), 243-251.
55. Svldronova, P. B. B., Vojtova, L. 2014. Crosslinking of Polysaccharide Microfibers. Master Thesis, Brno University of Technology, Brno.
56. Hatipo\check{g}lu, E. 2011. Ameliyat Sonrasi Karin Içi Yapişikliklarin Önlenmesinde Sodyum Hyaluronat Karboksimetilselüloz Membran, Polietilen Glikol—Lysine Ve Hyaluronik Asitin Etkinli\check{g}inin Wistar Albino Tipi Siçanlarda Yapilan Deneysel Çalişma Ile Araştirilmasi. Uzmanlik Tezi, Istanbul Üniversitesi, Istanbul.
57. Diamond, M. P., Linsky, C. B., Cunningham, T., Kamp, L., Pines, E., DeCherney, A. H. 1991. “Synergistic Effects of Interceed (TC7) and Heparin in Reducing Adhesion Formation in the Rabbit Uterine Horn Model”, Fertility and Sterility, 55(2), 389-394.
58. Hooker, G. D., Taylor, B. M., Driman, D. K. 1999. “Prevention of Adhesion Formation with Use of Sodium Hyaluronate-Based Bioresorbable Membrane in a Rat Model of Ventral Hernia Repair with Polypropylene Mesh-A Randomized, Controlled Study”, Surgery, 125(2), 211-216.
59. Andrady, A. L. 2008. Science and Technology of Polymer Nanofibers. New Jersey: Wiley Pres.
60. Chronakis, I. S. 2005. “Novel Nanocomposites and Nanoceramics Based on Polymer Nanofibers Using Electrospinning Process-A Review”, Journal of Materials Processing Technology, 167(2-3), 283-293.
61. Süpüren, G., Kanat, Z. E., Çay, A., Kirci, T., Gülümser, T., Tarakçio\check{g}lu, I. 2007. “Nano Lifler (Bölüm 2)”, Tekstil ve Konfeksiyon, 2, 83-89.
62. Doshi, J., Reneker, D. H. 1995. “Electrospinning Process and Applications of Electrospun Fibers”, Journal of Electrostatics, 35(2-3), 151-160.
63. Üstünda\check{g}, G. C., Karaca, E. 2009. “Poli(Vinil Alkol)/Sodyum Alginat Karigimlarindan Elektro Çekim Yöntemi Ile Elde Edilen Nanolifli Yüzeylerin Incelenmesi”, Uluda\check{g} Üniversitesi Mühendislik Mimarlik Fakültesi Dergisi, 14(1), 159-172.
64. Mit-uppatham, C., Nithitanakul, M., Supaphol, P. 2004. “Ultrafine Electrospun Polyamide-6 Fibers: Effect of Solution Conditions on Morphology and Average Fiber Diameter”, Macromolecular Chemistry and Physics, 205, 2327-2338.
65. Ramakrishna, S., Fujihara, K., Teo, W. E., Lim, T. C., Ma, Z. 2005. An Introduction to Electrospinning and Nanofibers. Singapore: World Scientific Publishing Co.
66. Deitzel, J., Kleinmeyer, J., Harris, D, Becktan, N. C. 2001. “The Effect of Processing Variables on the Morphology of Electrospun Nanofibers and Textiles”, Polymer, 42, 261-272.
67. Kozano\check{g}lu, G. S. 2006. Elektrospinning Yöntemiyle Nanolif Üretim Teknolojisi. Yüksek Lisans Tezi, Istanbul Teknik Üniversitesi, Istanbul.
68. Angammana, C. J., Jayaram, S. H. 2008. “Analysis of the Effects of Solution Conductivity on Electro-Spinning Process and Fiber Morphology”, Industry Applications Society Annual Meeting, Canada.
69. Li, Z, Wang, C. 2013. One-Dimensional Nanostructures Electrospinning Technique and Unique Nanofibers. New York: Springer.
70. Aşcio\check{g}lu, B. 2005. Manufacturing and Heat Transfer Analysis of Nano-Micro Fiber Composites. Ph.D Thesis, Auburn University, Auburn.

1. A nanofibrous mat obtained from a hyaluronic acid and sodium alginate polymer mixture, suitable for use as an adhesion barrier in the biomedical field.

2. A production method of the nanofibrous mat according to claim 1, the method comprising the following process steps:
preparation of the hyaluronic acid solution in a solvent;
preparation of the aqueous sodium alginate solution;
mixing of the two prepared solutions;
application of an electrospinning method to the mixed solution; and
cross-linking of the obtained nanofibrous mat.

3. A method according to claim 2, wherein the solvent is NaOH/Dimethyl sulfoxide or NaOH/Dimethyl formamide.

4. The method according to claim 3, wherein the ratio of the mixture of said NaOH with Dimethyl sulfoxide or NaOH to Dimethyl formamide is 4/1 by volume.

5. The method according to claim 2, wherein the concentration of hyaluronic acid solution prepared in the solvent is 8-15% by weight/volume %.

6. The method according to claim 2, wherein the concentration of sodium alginate solution prepared in distilled water is 1-4% by weight/weight %.

7. The method according to claim 2, wherein the hyaluronic acid solution and the sodium alginate solution are mixed at a ratio of 1/1-10/1 by volume.

8. The method according to claim 2, wherein the said cross-linking treatment comprises following process steps:
dissolving of 1-ethyl-3-(3-imethylaminopropyl) carbodiimide hydrochloride in a solvent;
dissolving of N-hydroxysuccinimide or divinyl sulfone in a solvent;
mixing of the two solutions prepared;
submerging of the nanofibrous mat, into the resulting mixture solution and waiting at room temperature for 24 hours;
agitation of the nanofibrous mats removed from the mixture solution in ethanol;
leaving in an incubator at 37° C. for 12 hours to dry.

9. The method according to claim 8, wherein the 1-ethyl-3-(3-imethylaminopropyl) carbodiimide hydrochloride, N-hydroxysuccinimide or divinyl sulfone is 50-100 mM.

10. The method according to claim 8, comprising the mixing of the two solutions prepared in a volume of 1/1-3/1 by volume.

11. The method according to claim 8, wherein the said solvent is ethanol or methanol.

12. A nanofibrous mat obtained from the hyaluronic acid and sodium alginate polymer mixture with the electrospinning method, suitable for use as an adhesion barrier in the biomedical field.
